(19) United States
(12) Patent Application Publication
(10) Pub. No.: US 2026/0021321 A1
(43) Pub. Date: Jan. 22, 2026

(54) PERSONAL MASK WITH COMBINATION STIMULATION LED

(71) Applicant: Y & J BIO Co., Ltd., Hanam-si, Gyeonggi-do (KR)

(72) Inventors: Young Sam CHUN, Goyang-si, Gyeonggi-do (KR); Hwa Sik JEONG, Anyang-si, Gyeonggi-do (KR)

(21) Appl. No.: 18/993,124

(22) PCT Filed: Jul. 11, 2023
(86) PCT No.: PCT/KR2023/009897

§ 371 Date: Jan. 10, 2025

(30) Foreign Application Priority Data
Jul. 15, 2022 (KR) 10-2022-0087668

(51) Int. Cl. A61N 5/06

(52) U.S. Cl. CPC A61N 5/0616; A61N 2005/0627; A61N 2005/0632; A61N 2005/0647; A61N 2005/0652; A61N 2005/0659; A61N 2005/0663

(57) ABSTRACT

An embodiment relates to a light emitting diode (LED) mask for personal combination stimulation, which is to be worn on a face, and more specifically to an LED mask for personal combination stimulation, which can prevent skin damage due to heat generation caused by light emission in an LED, reduce manufacturing and maintenance costs, and effectively make improvement such as whitening facial skin, reducing wrinkles, in particular, wrinkles around eyes. The LED mask for personal combination stimulation includes a mask unit to be put on the face of a user; and a light irradiation unit installed in the mask unit and including a plurality of LEDs arranged on a substrate, wherein the LEDs comprise multiple types of LED chips to emit light of different wavelengths, and the multiple types of LED chips are configured as a combination LED module in a single support structure and arrange on the substrate.

### TECHNICAL FIELD

The disclosure relates to a light emitting diode (LED) mask for personal combination stimulation, which is to be worn on a face, and more specifically to an LED mask for personal combination stimulation, which can prevent skin damage due to heat caused by light emission in an LED, reduce manufacturing and maintenance costs, and effectively make improvement actions such as whitening facial skin, reducing wrinkles, in particular, wrinkles around eyes.

### BACKGROUND ART

In general, phototherapy for skin beauty or skin disease treatment is based on the principle of promoting biochemical reactions within the skin of a human body by irradiating the skin with light from various light sources such as lasers, fluorescent lamps, and ultraviolet lamps, and is used for the treatment of damaged skin, cosmetic purposes such as whitening, etc. through selective regeneration or destruction of skin tissue.

In particular, light emitting diode (LED) light sources, unlike high-power laser light sources, may output light suitable for a human body, which is irradiated to a wide cosmetic or treatment area, and the light irradiated in this way may induce a photobiochemical reaction between skin cells similarly to the principle that sunlight is converted into plant cells through chlorophyll in plants.

In addition, the light-emitting diode (LED) light sources are based on the fact that photons are received by chromatophores or photoreceptors in cell tissues and promote cellular metabolic activities, in which the light absorbed in cells increases reactive oxygen species (ROS) and adenosine triphosphate (ATP) synthesis in the cell tissues. For example, cells exposed to red light and near-infrared light may release nitric oxide (NO), etc., and these released components may undergo photolysis and promote enzyme activation.

Accordingly, when wearing a beauty mask mounted with multiple LED light sources, the LED light sources may activate the overall metabolic activity of cells and play a major role in cell regeneration and rehabilitation, thereby promoting the production of collagen and elastin in the skin, suppressing the occurrence of wrinkles, and keeping the skin moist and elastic.

For example, Korean Patent No. 10-2223108 has proposed an “LED mask” as the foregoing beauty mask.

The conventional LED mask 1 described above includes an inner mask unit 100 to be worn on the face of a user, and a charging unit 200 to charge and sterilize the inner mask unit 100.

The inner mask unit 100 includes a mask body 110 to be put on the face of a user, a light emitting unit 120 provided inside the mask body 110 to irradiate light having different wavelengths to the face of a user, a nasolabial wrinkle position adjusting member 130 detachably coupled to the inside of the mask body 110 to adjust the range of light irradiated to the nose and lips of a user, and a fastening belt 140 for securing the mask body 110 to the head of a user.

The mask body 110 supports LEDs 123, 125, 127, and 129 of the light-emitting unit 120, which are arranged to cover the face of a user and accommodated therein so that light emitted therefrom can be irradiated to the face of a user. As shown in FIG. 1, the mask body 110 includes an outer mask 111, and an inner mask 113 provided on the inner side of the outer mask 111. The light emitting unit 120 is accommodated between the outer mask 111 and the inner mask 113.

The inner mask 113 is coupled to the inner side of the outer mask 111 to support the light-emitting unit 120, and transmits light emitted from the LEDs 123, 125, 127 and 129 of the light-emitting unit 120 so that light can be irradiated to the face. The inner mask 113 is formed of a material that transmits light, and is provided with an inner eye-exposure hole 113a, a nose insertion groove 113b, an inner nose breathing hole 113c, and an inner mouth breathing hole 113d at the same positions as those of the outer mask 111.

In addition, a silicone member 115 is provided around the inner eye-exposure hole 113a so that the inner mask 113 can be seated on the face of a user. The silicone member 115 is formed to protrude to a certain height, thereby preventing light emitted from the LEDs 123, 125, 127 and 129 from entering the eyes of a user.

To prevent light from being irradiated to a wearer's eyes, the conventional LED mask includes an oval-shaped silicone member 115 installed in the inner mask 113. However, the silicone member 115 is formed of an opaque silicone material to block incident light, and thus incident light for improving wrinkles on skin around the eyes is also blocked.

Accordingly, there is a disadvantage in that any improvement in reducing wrinkles in eye areas, such as an area between eyebrows, eyelids, and the corners of the eyes, in which wrinkles occur severely, is not expected.

In addition, the conventional LED mask as disclosed in Korean Patent No. 10-2223108 has asserted its effects on whitening the skin and improving wrinkles, but there are few cases of actual verification or application. Further, the reality is that the conventional LED mask is vaguely based on some research results showing that LEDs are effective in whitening the skin and improving wrinkles.

In particular, the conventional LED mask has the disadvantage of causing skin damage and, in severe cases, burns due to excessive heat generation as the number of LEDs to be installed is indiscriminately increased based only on research results showing that the skin improvement effect increases in proportion to the quantity of light. Further, the conventional LED mask has limitations involving various problems such as increased battery capacity due to an excessive number of LEDs, increased component costs due to additional components and the increased capacity, increased power consumption, etc.

In addition, although conventional LED technology has been described as using LEDs that are effective in improving wrinkles, the reality is that the LEDs actually used in mask-type products cannot produce high optical output like medical devices used in hospitals because the power of a built-in battery is limited. Therefore, there are limitations in that the optical output of actual products is insufficient to expect the wrinkle improvement effect.

### DOCUMENT OF RELATED ART

### Patent Document

(Patent Document 1) Korean Patent No. 10-2223108, titled “LED MASK”
(Patent Document 2) Korean Patent Publication No. 10-2021-0058295, titled “LED MASK”

### DISCLOSURE

### Technical Problem

The disclosure has been conceived to solve the problems described above, and an aspect of the disclosure is to provide a light emitting diode (LED) mask for personal combination stimulation, in which improvement functions such as whitening facial skin and reducing wrinkles are substantially implemented by using a combination stimulation LED where LED chips of various wavelength bands are combined.

Another aspect of the disclosure is to provide an LED mask for personal combination stimulation, which has an LED structure and an LED layout structure to prevent skin damage due to heat from an LED while effectively performing skin whitening and wrinkle improvement.

Still, another aspect of the disclosure is to provide an LED mask for personal combination stimulation, which can effectively improve wrinkles around the eyes while blocking light from entering the eyes.

### Technical Solution

According to an embodiment of the disclosure, a light emitting diode (LED) mask for personal combination stimulation, including: a mask unit to be put on the face of a user; and a light irradiation unit installed in the mask unit and including a plurality of LEDs arranged on a substrate, wherein the LEDs include multiple types of LED chips to emit light of different wavelengths, and the multiple types of LED chips are configured as a combination LED module in a single support structure and arrange on the substrate.

The combination LED module may include a first LED chip for a wavelength of 625 to 635 nm, and a second LED chip for a wavelength of 845 to 855 nm, which are configured in the single support structure.

The first LED chip may have a light irradiance output of 10 mW/cm²±20% for a wavelength of 630 nm, and the second LED chip may have a light irradiance output of 10 mW/cm²±20% for a wavelength of 850 nm.

The number of combination LED modules to be arranged ensures that the internal temperature of the mask unit rising due to heat generation caused by light emission does not exceed 41 to 43° C., and a distance between the combination LED module to be installed in the mask unit and the surface of the skin to be cared ensures that 50% of the light irradiation output is irradiated to the skin surface.

The combination LED module may have a light irradiation angle of 120°, and the distance between the combination LED module 42 and the surface of the skin to be cared may be 10 mm±10%.

The combination LED modules may be installed on the substrate at intervals of 14 to 20 mm from each other taking a curvature of a facial area into account while ensuring that the internal temperature of the mask unit m does not exceed 41 to 43° C.

The mask unit may include an inner mask unit put on a user's face, formed with an inner eye-exposure hole, and including the light irradiation unit disposed on an outer surface, and an outer mask unit coupled to the inner mask unit and formed with an outer eye-exposure hole communicating with the inner eye-exposure hole.

The LED mask may further include: a light-blocking member installed around the inner eye-exposure hole and the outer eye-exposure hole to prevent light from entering the eyes; and a light transfer supply means installed in the inner mask unit to irradiate skin around the eyes with light, wherein the light transfer supply means includes a light supply unit installed around the inner eye-exposure hole and formed of a light-transmitting material to make light enter and exit, and a light transfer unit arranged between the light supply unit and the LED to transfer light to the light supply unit.

The light supply unit may include a flexible light-transmitting pad seated on the inner side of the light-blocking member and having a communication hole to communicate with the inner eye-exposure hole, a light guide tube connecting portion formed on the flexible light-transmitting pad and having a fitting hole, and a plurality of light supply line portions formed on the flexible light-transmitting pad to move and diffuse light introduced through the light transfer unit.

The light transfer unit may include a light guide tube having a first end disposed to face the LED of the light irradiation unit, and a second end connected to the fitting hole.

The light-blocking member may include an inner light-blocking unit formed in a substantially oval closed curve shape to be seated inside the circumference of the inner eye-exposure hole and having a lower center portion from which a nose-mounting portion having a nose-mounting groove is extended, and an outer light-blocking unit extended from the circumference of the inner light-blocking member at an acute angle to the inner light-blocking member and formed with a fitting protrusion at an edge thereof to be fitted to a gap between the inner eye-exposure hole and the outer eye-exposure hole

Preferably, the flexible light-transmitting pad may include a seating pad portion arranged to surround an outer portion of the inner light-blocking unit, formed as a roughly oval body provided with a nose insertion cut portion to be seated around an eye area upon being worn, and including a seating protrusion formed along an inner edge thereof to allow the edge of the inner light blocking unit to be seated thereon; a front support portion extending downward on the seating pad portion being in contact with the nose insertion cut portion; a light guide tube installation portion bent at a bottom of the front support portion and formed with a light guide tube insertion port; and a cushion connection portion formed extending along an outer periphery of the seating pad portion, connected to the front support portion and fastened to the inner mask unit.

The light supply line portion may include an eyelid light supply line portion formed on the seating pad portion corresponding to both eyelids and end corners of the eyes to scatter and diffuse light, and an inter-eyebrow light supply line portion formed on the seating pad portion corresponding to an area between eyebrows.

The eyelid light supply line portion may be formed as multiple light-transmitting protrusion lines arranged in parallel from the surface of the seating pad portion being in contact with the nose insertion cut portion toward a second side.

The inter-eyebrow light supply line portion may be formed as multiple light-transmitting protruding lines repeatedly protruding in arc shapes from the seating pad portion corresponding to the inter-brow area and different in radius of curvature from each other.

### Advantageous Effects

An LED mask for personal combination stimulation according to the disclosure employs a combination stimulation LED module, in which an LED chip having a light irradiance output of 10 mW/cm²±20% for a wavelength of 630 nm and an LED chip having a light irradiance output of 10 mW/cm²±20% for a wavelength of 850 nm are combined to irradiate light to skin to be cared with 50% of the light irradiance output at a distance of 10 mm, thereby effectively performing skin whitening effects and wrinkle reduction effects.

In the LED mask for personal combination stimulation according to the disclosure, an LED structure and an LED layout structure capable of preventing skin damage caused by heat generation of LEDs are implemented to minimize the battery capacity and the number of LEDs, thereby improving safety and durability in use and having effects on reducing manufacturing and maintenance costs.

Further, the LED mask for personal combination stimulation according to the disclosure includes a light-blocking member for blocking light from entering the eyes and a light transfer supply means provided in the inner eye-exposure hole of the inner mask unit and having flexibility and light-transmitting properties to irradiate skin around an eye area with light even though the LED mask is in close contact with the eye area, thereby having an effect on performing wrinkle improvement.

### DESCRIPTION OF DRAWINGS

FIG. 1 is a view for illustrating a conventional light emitting diode (LED) mask.

FIG. 2 is a perspective view showing an LED mask for personal combination stimulation according to an embodiment of the disclosure.

FIG. 3 is an exploded perspective view showing an LED mask for personal combination stimulation according to an embodiment of the disclosure.

FIG. 4 is a plan view showing a light irradiation unit of an LED mask for personal combination stimulation according to an embodiment of the disclosure.

FIG. 5 is a view for illustrating an installation structure of a combination LED module arranged in a light irradiation unit of an LED mask for personal combination stimulation according to an embodiment of the disclosure.

FIG. 6 is a view schematically showing light irradiation areas of a combination LED module arranged in a light irradiation unit of an LED mask for personal combination stimulation according to an embodiment of the disclosure.

FIG. 7 is a perspective view showing a shape appearing inside an LED mask for personal combination stimulation according to an embodiment of the disclosure.

FIGS. 8A and 8B are perspective views showing eye areas of an LED mask for personal combination stimulation according to an embodiment of the disclosure, in which FIG. 8A shows a shape appearing on the outer side, and FIG. 8B shows a shape appearing on the inner side.

FIG. 9 is an enlarged perspective view for illustrating a nasolabial wrinkle improvement portion of an LED mask for personal combination stimulation according to an embodiment of the disclosure.

FIGS. 10A and 10B are views for illustrating a light-blocking member of an LED mask for personal combination stimulation according to an embodiment of the disclosure, in which FIG. 10A shows a shape appearing on the first side, and FIG. 10B shows a shape appearing on the second side.

FIGS. 11A and 11B are views for illustrating a light transfer supply means of an LED mask for personal combination stimulation according to an embodiment of the disclosure, in which FIG. 11A shows a shape appearing on the first side, and FIG. 11B shows a shape appearing on the second side.

FIG. 12A is a cutaway perspective view taken along line A-A in FIG. 2, and FIG. 12B is a cutaway perspective view of B in FIG. 2.

FIG. 13 is a view for illustrating the use state of an LED mask for personal combination stimulation according to an embodiment of the disclosure.

FIG. 14 is a graph showing a degree of improvement in the crow's feet grading scale (CFGS) by comparing a proportion of subjects with improved wrinkles around their eyes as a result of clinical trials of an LED mask for personal combination stimulation according to an embodiment of the disclosure.

FIG. 15 is a graph showing a degree of improvement in the global aesthetic improvement scale (GAIS) of an eye area by comparing a proportion of subjects with improved wrinkles around their eyes as a result of clinical trials of an LED mask for personal combination stimulation according to an embodiment of the disclosure.

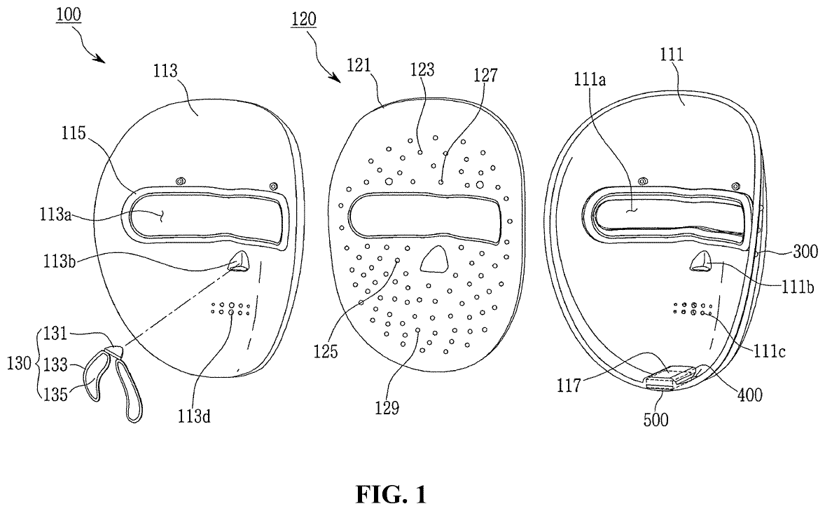

### MODE FOR INVENTION

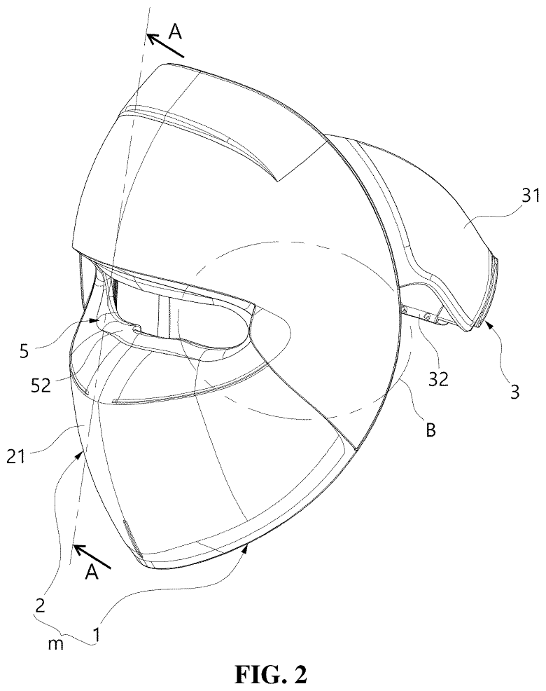

Below, exemplary embodiments of the disclosure will be described in detail with reference to the accompanying drawings FIGS. 2 to 15, in which like numerals refer to like elements throughout FIGS. 2 to 15.

Meanwhile, detailed descriptions of the configurations and their operations and effects, which can be easily understood by a person having ordinary knowledge in the art from general technologies in the accompanying drawings, will be simplified or omitted. Further, the disclosure is characterized in a light emitting diode (LED) mask for personal combination stimulation, and thus parts related to those characteristics will be illustrated and described and the descriptions about the remaining parts will be simplified or omitted.

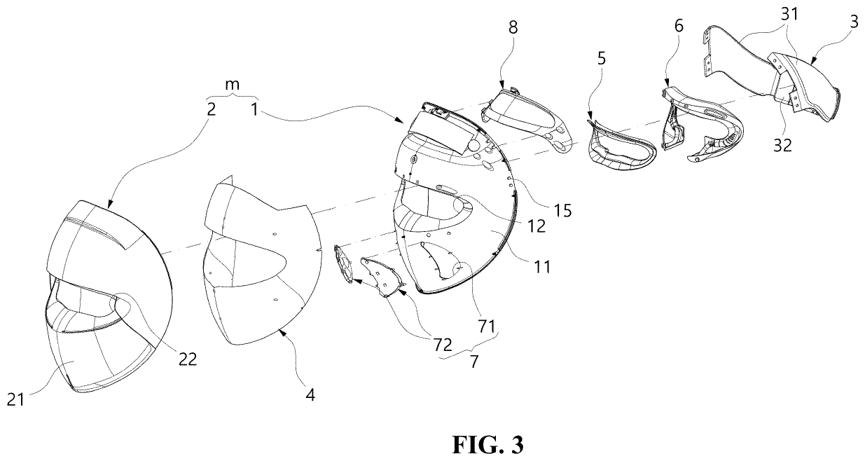

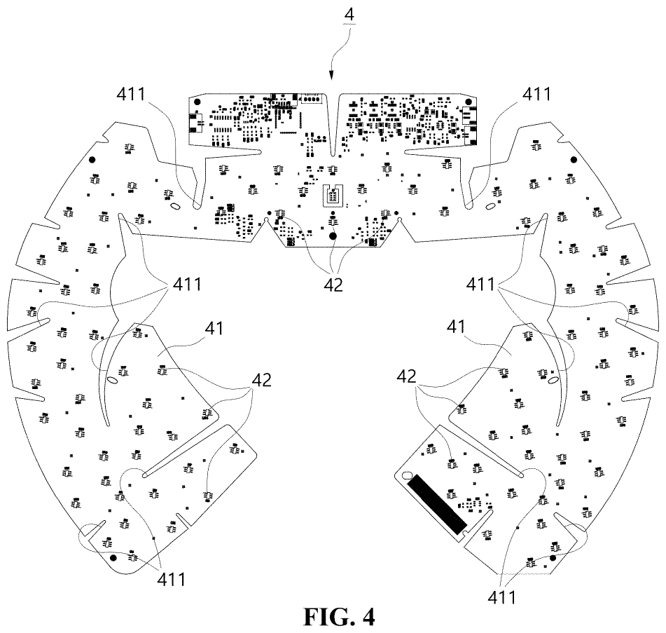

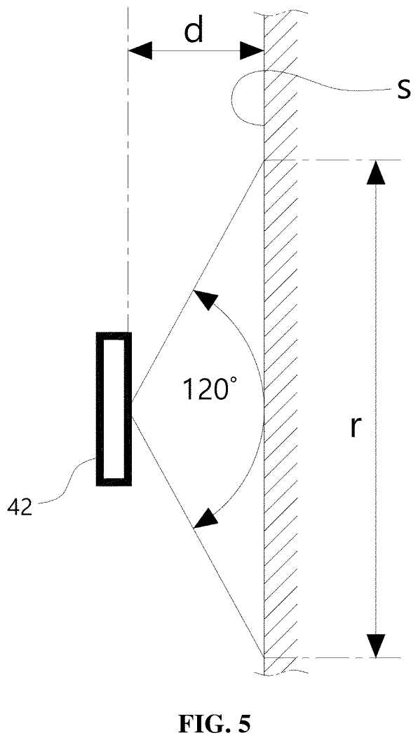

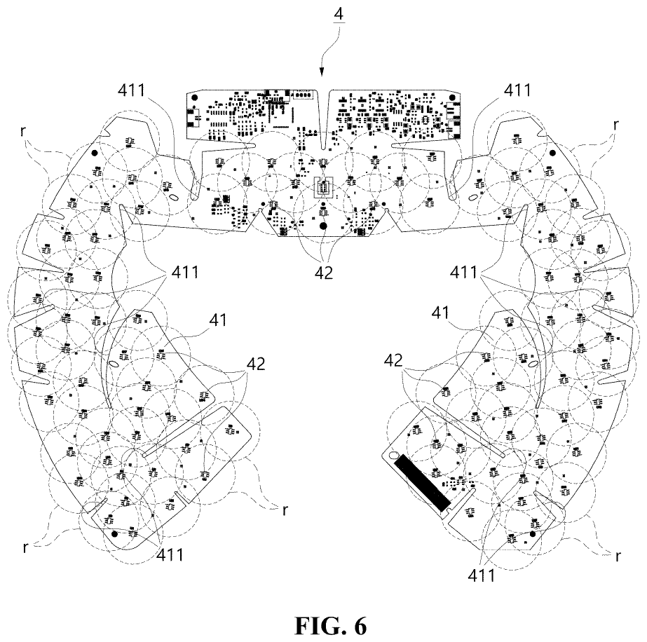

FIG. 2 is a perspective view showing the outer appearance of an LED mask for personal combination stimulation according to an embodiment of the disclosure. FIG. 3 is an exploded perspective view showing an LED mask for personal combination stimulation according to an embodiment of the disclosure. FIG. 4 is a plan view showing a light irradiation unit of an LED mask for personal combination stimulation according to an embodiment of the disclosure. FIG. 5 is a view for illustrating an installation structure of a combination LED module arranged in a light irradiation unit of an LED mask for personal combination stimulation according to an embodiment of the disclosure. FIG. 6 is a view schematically showing light irradiation areas of a combination LED module arranged in a light irradiation unit of an LED mask for personal combination stimulation according to an embodiment of the disclosure.

Referring to FIGS. 2 to 6, the LED mask for personal combination stimulation according to an embodiment of the disclosure is characterized in using a combination stimulation LED, in which LED chips of various wavelength bands are combined, not only to make improvement effects such as whitening facial skin and reducing wrinkles but also to prevent skin damage caused by heat from the LED, and includes a mask unit m to be put on a user's face, and a light irradiation unit 4 to be installed in the mask unit m.

The mask unit m may be configured as a single mask structure without any special limitations to the shape or structure as long as it is structured to be worn on a user's face. However, in this embodiment, as described specifically below, the mask unit m may be divided into an inner mask unit 1 located on the inner side and an outer mask unit 2 located on the outer side of the inner mask in consideration of the installation stability, assembling ability, and use convenience of the light irradiation unit 4.

The light irradiation unit 4 is installed in the mask unit m as illustrated in FIG. 4 and has a plurality of LEDs arranged on a substrate 41. The light irradiation unit 4 may be configured with LEDs of various wavelength bands without any special limitations. However, this embodiment is characterized in that the LED chips of various wavelength bands are combined to be suitable for skin care, such as wrinkle improvement and whitening effects through tests of irradiating the skin in various ways.

In more detail, the light irradiation unit 4 has main characteristics in that the combination LED module 42 where multiple types of LED chips emitting light of different wavelengths are integrally configured as a single support structure is placed on the substrate 41.

The combination LED module 42 includes a first LED chip (not shown) having a light irradiance output of 10 mW/cm²±20% for a wavelength of 625 to 635 nm, and a second LED chip (not shown) having a light irradiance output of 10 mW/cm²±20% for a wavelength of 845 to 855 nm, which are configured as a single support structure to implement combined optical stimulation (hereinafter referred to as combination stimulation).

In particular, optimal conditions for skin care were achieved when the first LED chip had a light irradiance output of 10 mW/cm²±20% for a wavelength of 630 nm, and the second LED chip had a light irradiance output of 10 mW/cm²±20% for a wavelength of 850 nm. For example, the combination LED module may include RED & IR DUAL TOP VIEW LED (size: 3.5×2.8×1.8 mm) including 630 nm RED LED chips and 850 nm IR LED chips from domestic and foreign LED manufacturers.

In addition, some research results show that the more the combination LED modules 42 installed, the more advantageous it is for skin care. However, if the number of LEDs installed is increased excessively, it may cause serious skin damage due to excessive heat generation, and cause various problems due to the increase in the number of LEDs, increase in battery capacity, etc. Therefore, it is important to configure the LED structure and LED layout structure so that the internal temperature of the mask unit does not exceed 41 to 43° C. Here, the internal temperature of the mask unit may be 41° C. or lower. In addition, the mask unit may be allowed to have an internal temperature of 41 to 43° C. by the medical device approval agency.

Referring to FIGS. 3 to 5, the LED mask for personal combination stimulation according to an embodiment of the disclosure shows that the number of combination LED modules 42 and a distance d at which a light irradiation output of the combination LED module 42 is irradiated to a skin surface are important to prevent the internal temperature of the mask unit m from rising to a high temperature due to heat caused by light emission and to prevent adverse effects on skin, which are based on various tests.

Based on these facts, according to this embodiment, the combination LED modules 42 arranged on the mask unit m needs to be set in quantity to such an extent that the internal temperature of the mask unit m rising due to heat generation does not exceed 41 to 43° C., and needs to be installed at a distance d such that 50% of the light irradiation output is irradiated to the skin to be cared.

In more detail, to satisfy the conditions of ensuring that 50% of the light irradiation output is irradiated to a skin surface s to be cared while the internal temperature of the mask unit m does not exceed 41 to 43° C., the combination LED module 42 may be configured to have a light irradiation angle of 120° and a distance between the combination LED module 42 and the skin s to be cared may be 10 mm+10%.

In addition, the combination LED modules 42 are installed on the substrate 41 at intervals of 14 to 20 mm from each other taking the curvature of the facial area into account while ensuring that the internal temperature of the mask unit m does not exceed 41 to 43° C. The desirability of this condition was confirmed through numerous comparative tests (details omitted) using various intervals. In this way, when the intervals between the combination LED modules 42 range from 14 to 20 mm, an intersection area (crossing area) formed between circular light irradiation areas r as shown in FIG. 6, and a light quantity in this intersection area is relatively large to naturally irradiate a stable quantity of light even to a curved portion of the face, thereby enabling appropriate skin care.

Meanwhile, the light irradiation unit 4 is arranged between the outer surface of the inner mask unit 1 and the inner surface of the outer mask unit 2, which will be described later, and is disposed to irradiate light toward a light transfer supply means 6 and the inner mask unit.

The substrate 41 is formed as a thin film structure, and includes a plurality of terminals to which a plurality of combination LED modules 42 are connected, and circuits connected to the terminals, etc. mounted thereto. In addition, the substrate 41 provided in the form of a film has a plurality of cut portions 411 formed thereon so as to be easily placed close to the inner mask unit 1 formed with a plurality of curved portions to fit the shape of a face with the eyes, nose, mouth, ears, and cheekbones, etc.

Below, the mask unit m according to the disclosure will be described in detail.

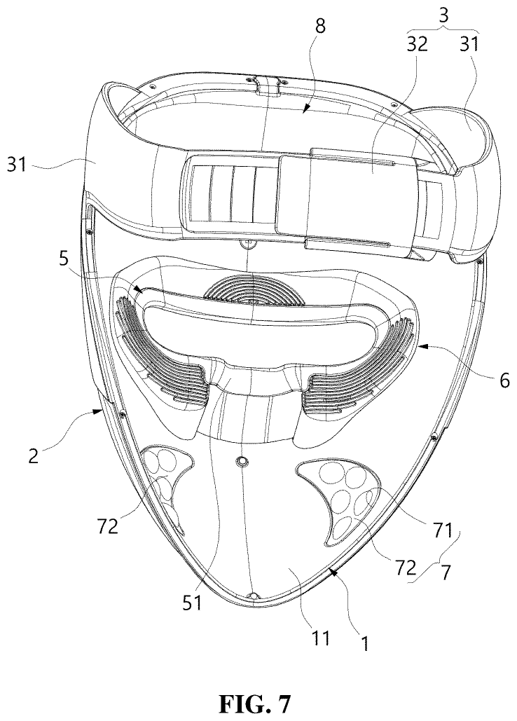

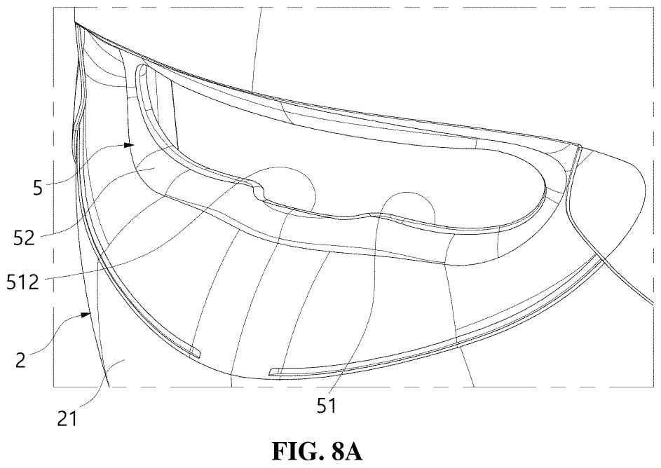

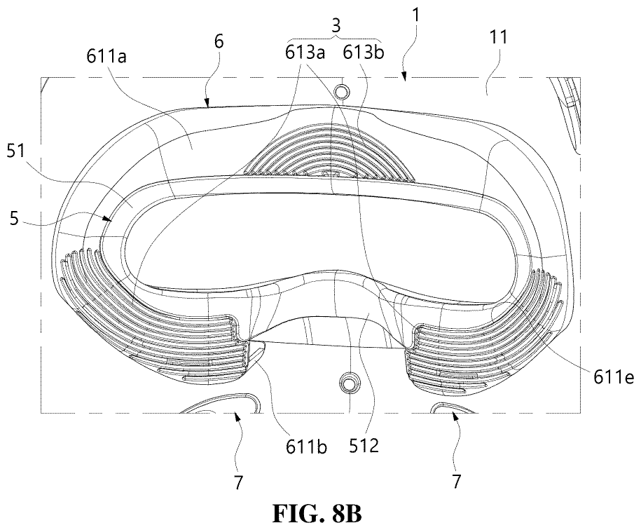

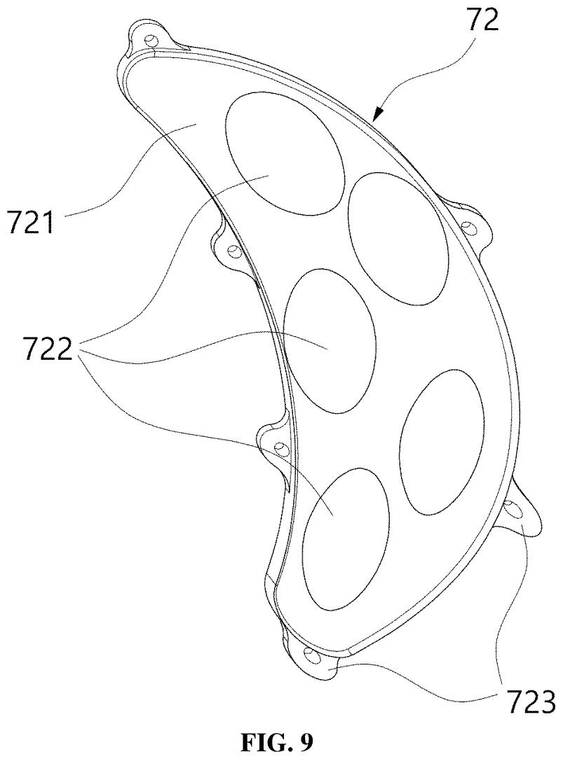

FIG. 7 is a perspective view showing a shape appearing inside an LED mask for personal combination stimulation according to an embodiment of the disclosure, and FIGS. 8A and 8B are perspective views showing eye areas of an LED mask for personal combination stimulation according to an embodiment of the disclosure, in which FIG. 8A shows a shape appearing on the outer side, and FIG. 8B shows a shape appearing on the inner side. FIG. 9 is an enlarged perspective view for illustrating a nasolabial wrinkle improvement portion of an LED mask for personal combination stimulation according to an embodiment of the disclosure.

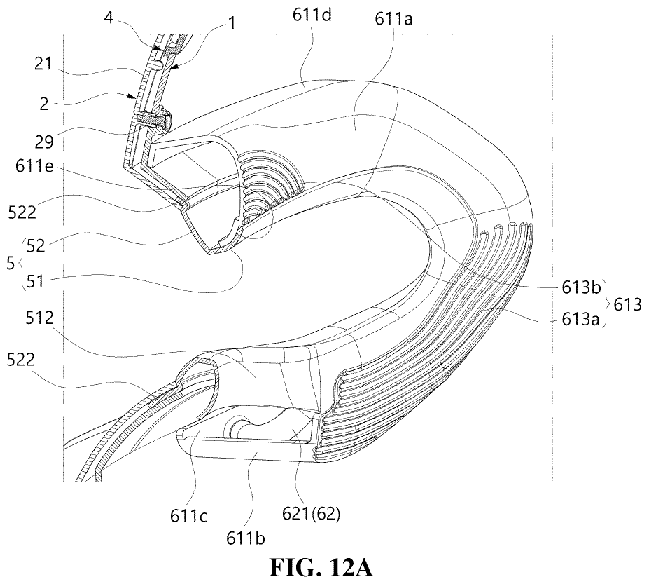

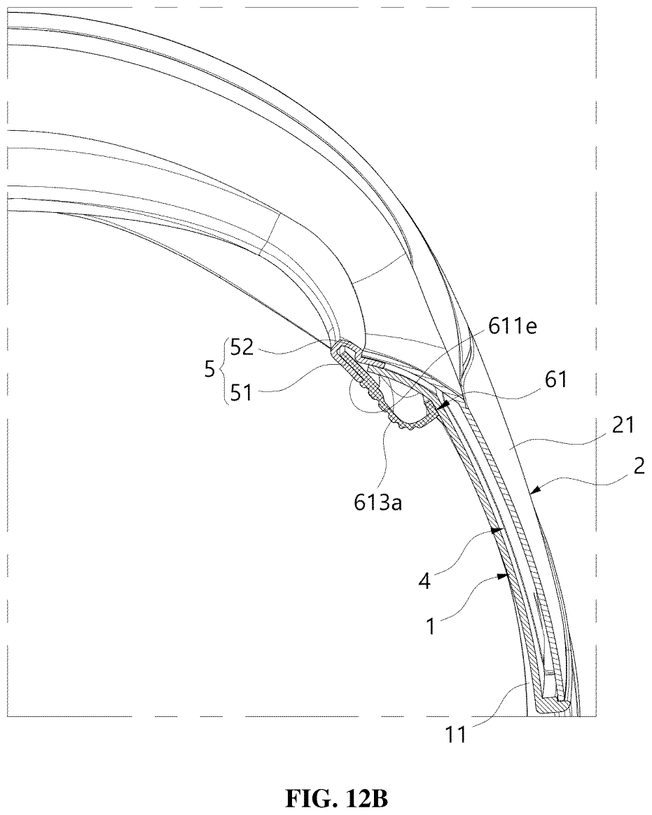

Referring to FIGS. 2, 3, 7 to 8B, the inner mask unit 1 is shaped to cover the face of a human body, has a structure in which an inner eye-exposure hole 12 is perforated in an inner mask body 11 to be worn on a user's face, and is formed with a fastening band fixing portion 15 for fastening a wearing fixing means 3, and a fastening hole (with no reference numeral) for coupling with a fastening boss 29 (see FIG. 12A of the outer mask unit 2, etc. are formed.

Further, the inner mask unit 1 is formed with nasolabial wrinkle improvement portions 7 so that light for nasolabial wrinkle improvement is diffused and incident to locations corresponding to nasolabial wrinkle formation areas on both sides of a face.

The nasolabial wrinkle improvement portions 7 are each roughly shaped like a crescent moon, are formed on both lower sides of the inner mask body 11, and include a wrinkle improvement plate installation hole 71 perforated in the nasolabial wrinkle formation areas of a face, and a wrinkle improvement plate 72 coupled to the wrinkle improvement plate installation hole 71.

The wrinkle improvement plate 72 includes a plurality of diffusion lenses 722 protruding from a light-transmitting plate-shaped body 721 to diffuse light. The wrinkle improvement plate 72 is formed with an assembly protrusion 723 having a protrusion insertion groove into which a plurality of coupling protrusions (not shown) protruding from the circumferential surface of the wrinkle improvement plate installation hole 71 are inserted. Further, a substrate (not shown) provided in the form of a film, on which the LEDs emitting light for the nasolabial wrinkle improvement is installed, is provided on the inner surface of the wrinkle improvement plate 72.

In addition, the wrinkle improvement plate may be configured in various forms other than the aforementioned structure as long as it can diffuse and supply light. Although not shown, a plurality of light supply lines protruding in the form of linear protrusions for light diffusion may be densely formed on the light-transmitting plate-shaped body.

Meanwhile, a forehead seating member 8, in which the forehead of a user is seated when worn, is provided in an upper portion of the inner mask unit 1. The forehead seating member 8 may be formed of a material or structure having cushioning characteristics so as not to cause pain to a user while maintaining a stable wearing state.

The outer mask unit 2 is shaped corresponding to the outer surface of the inner mask unit 1 so as to couple with the inner mask unit 1, and includes an outer eye-exposure hole 22 formed in an outer mask body 21, which is formed corresponding to the inner mask unit 1 to be covered with the light irradiation unit 4, and communicating with the inner eye-exposure hole 12.

In addition, the outer mask unit 2 may be configured as a single injection-molded unit or a plurality of injection-molded units, and includes a fastening boss 29 (see FIG. 12A) formed on the inner surface thereof to couple with the inner mask unit 1.

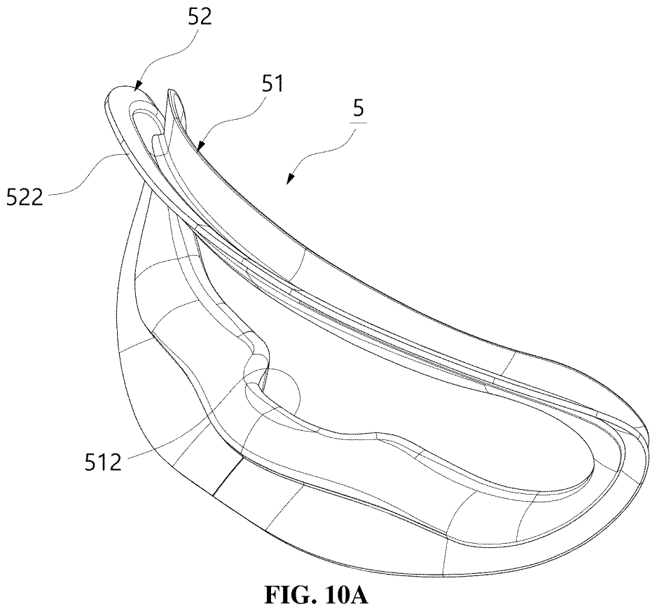

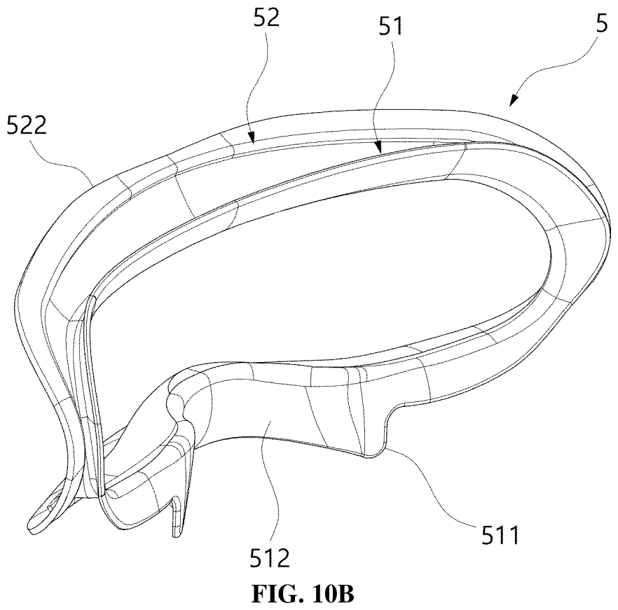

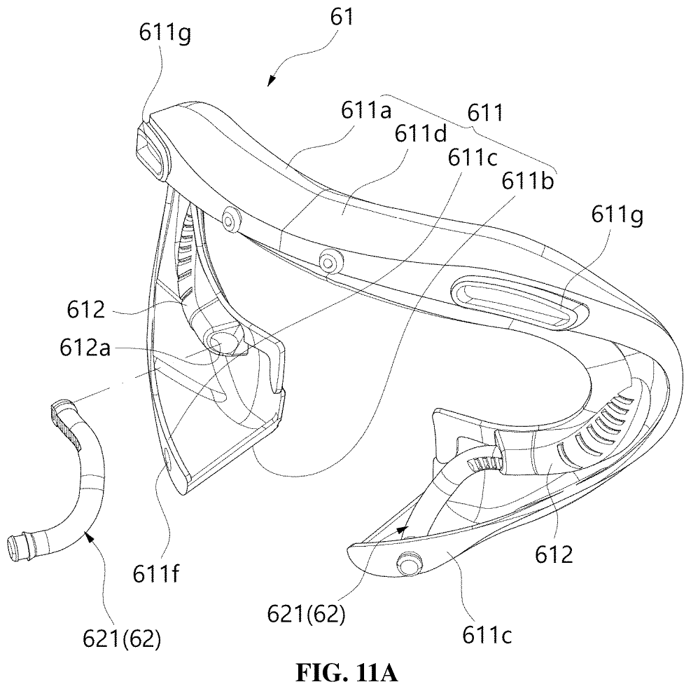

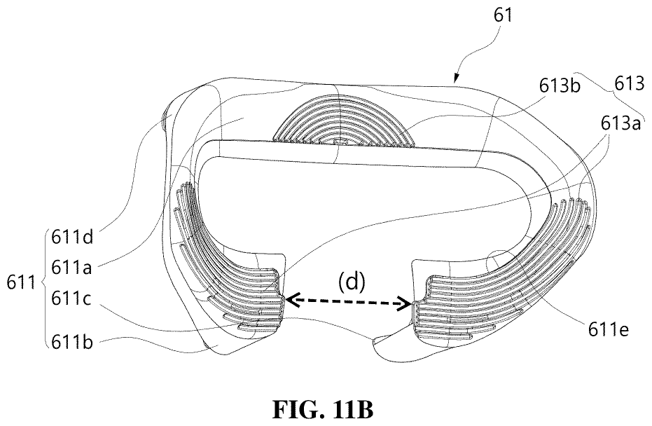

FIGS. 10A and 10b are views for illustrating a light-blocking member of an LED mask for personal combination stimulation according to an embodiment of the disclosure, in which FIG. 10A shows a shape appearing on the first side, and FIG. 10B shows a shape appearing on the second side. FIGS. 11A and 11B are views for illustrating a light transfer supply means of an LED mask for personal combination stimulation according to an embodiment of the disclosure, in which

FIG. 11A shows a shape appearing on the first side, and FIG. 11B shows a shape appearing on a second side. FIG. 12A is a cutaway perspective view taken along line A-A in FIG. 2, and FIG. 12B is a cutaway perspective view of B in FIG. 2.

Referring to FIG. 10A to 12B, the LED mask for personal combination stimulation according to the disclosure includes a wearing fixing means 3 for fixing the mask unit, a light-blocking member 5 for preventing light of the light irradiation unit 4 from entering an eye area, and a light transfer supply means 6 for irradiating light to skin around the eyes.

The wearing fixing means 3 refers to a component for fixing a skin care mask to the head of a human body when worn. Although there are no special limitations on the shape or structure of the wearing fixing means 3 as long as the wearing fixing means 3 can stably maintain the mask in a wearing state, the wearing fixing means 3 in this embodiment is configured as a fastening band to be fastened to the fastening band fixing portion 15 of the inner mask unit 1.

The fastening band may be configured as a publicly known fastening band adjustable in length to be worn on heads of various sizes. For example, the fastening band includes a band 31 connected to both fastening band fixing portions 15 of the inner mask unit 1, and a buckle 32 coupled to the band 31 to adjust the length of the band.

Referring to FIGS. 10A to 12B, the light-blocking member 5 refers to a component for blocking light generated by the LEDs from entering the eyes while a user wears the inner mask unit 1. The light-blocking member 5 is formed of a flexible and adhesive opaque material, through which light is not transmitted, and is installed around the inner eye-exposure hole 12 and the outer eye-exposure hole 22. For example, the light-blocking member 5 may be formed of opaque silicone rubber.

In more detail, as shown in FIGS. 10A to 10B, the light-blocking member 5 includes an inner light-blocking unit 51 formed in a substantially oval closed curve shape to be seated inside the circumference of the inner eye-exposure hole 12 and having a lower center portion from which a nose-mounting portion 511 having a nose-mounting groove 512 is extended, and an outer light-blocking unit 52 extended from the circumference of the inner light-blocking unit 51 at an acute angle (in a cross-section) to the inner light-blocking unit 51 and having a fitting protrusion 522 formed at the edge thereof to be fitted to a gap between the inner eye-exposure hole 12 and the outer eye-exposure hole 22.

Meanwhile, the light transfer supply means 6 is configured to make light having an improvement effect on reducing wrinkles enter around the eyes, and is installed in the inner mask unit 1 to irradiate the light to the skin around the eyes.

In more detail, the light transfer supply means 6 includes a light supply unit 61 installed around the inner eye-exposure hole 12 as shown in FIGS. 11A to 12B and formed of a light-transmitting material so as to make light enter and exit, and a light transfer unit 62 arranged between the light supply unit 61 and the LED (not shown) so as to transfer light to the light supply unit 61.

The light supply unit 61 includes a flexible light-transmitting pad 611 seated on the inner side of the light-blocking member 5 and having a communication hole communicating with the inner eye-exposure hole 12, a light guide tube connecting portion 612 formed on the flexible light-transmitting pad 611, and a plurality of light supply line portions 613 formed on the flexible light-transmitting pad 611 to move and diffuse light introduced through the light transfer unit 62.

Here, the light supply unit 61 may be made of medical silicone rubber or the like material without any specific limitations as long as it has light transmittance, flexibility, and cushioning characteristics. However, the light supply unit 61 in this embodiment is formed of a material called medical thermoplastic polyurethane (TPU). Such medical TPU has better biocompatibility and stability and better mechanical properties and processing properties than the existing medical materials such as PE, PP, TPE, PVC, and silicone rubber.

The flexible light-transmitting pad 611 includes a seating pad portion 611a to be in close contact with an eye area when worn, a front support portion 611b formed on the seating pad portion 611a, a light guide tube installation portion 611c, and a cushion connection portion 611d.

The seating pad portion 611a is arranged to surround the outer portion of the inner light-blocking unit 51, is formed as a roughly oval body to be seated around the eye area when worn, and provided with a nose insertion cut portion d cut in the lower center, and a seating protrusion 611e is formed along the inner edge thereof to allow the edge of the inner light blocking unit 51 to be seated thereon.

The front support portions 611b extend downward on both sides of the seating pad portion 611a being in contact with the nose insertion cut portion d so as to provide a space in which a light guide tube 621 is placed.

The light guide tube installation portion 611c is bent inwardly in the form of a protrusion at the bottom of the front support portion 611b and is formed with a light guide tube insertion port 611f.

The cushion connection portion 611d is formed extending along the outer periphery of the seating pad portion 611a and connected to the front support portion 611b, and includes a projection 611g to be fastened to the fastening hole of the inner mask unit 1. In addition, the cushion connection portion 611d is integrally extended from the edge of the seating pad portion so that the cross-sectional shape can have an approximately “L”-shape to provide cushioning when worn.

The light guide tube connecting portion 612 is configured as a tube connection projection that protrudes from the bottom surface of the seating pad portion 611a of the flexible light-transmitting pad 611. The tube connection projection has a fitting hole 612a recessed in the front thereof into which the light guide tube 621 is fitted, and is shaped to have the height gradually decreasing from the entrance of the fitting hole 612a rearwards.

The light supply line portion 613 includes an eyelid light supply line portion 613a formed on the seating pad portion 611a corresponding to both eyelids and end corners of the eyes to move, scatter and diffuse light, and an inter-eyebrow light supply line portion 613b formed on the seating pad portion 611a corresponding to an area between eyebrows to move, scatter and diffuse light.

The eyelid light supply line portion 613a is formed as multiple light-transmitting protrusion lines arranged in parallel from the end surface of the seating pad portion 611a being in contact with the nose insertion cut portion d toward the second side.

The inter-eyebrow light supply line portion 613b is formed as multiple light-transmitting protruding lines repeatedly protruding in arc shapes from the seating pad portion 611a corresponding to the inter-brow area and different in the radius of curvature. In this case, the radii of curvature of the light-transmitting protruding lines increase gradually from the inner side toward the outer side.

Meanwhile, the light transfer unit 62 includes the light guide tube 621 having a first end disposed to face the LED of the light irradiation unit 4 and a second end connected to the light guide tube connecting portion 612.

The light guide tube 621 is formed as a light-transmitting round rod structure, which has a first end connected to the light guide tube insertion hole (not shown) provided in the inner mask unit 1 via the light guide tube insertion port 611f of the light guide tube installation portion 611c and disposed to face the LED of the opposite substrate 41, and a second end formed in a curved shape to be inserted into the fitting hole 612a of the light guide tube connecting portion 612. Here, the light guide tube 621 may have an outer layer formed on its outer surface to prevent leakage of light during the movement of the light.

Meanwhile, the LED mask for personal combination stimulation according to an embodiment of the disclosure may include an input unit (not shown) such as a button for a user to control power on/off, select a use mode, input use time, etc.; a power supply unit (not shown) such as a secondary battery for supplying power to the LEDs, etc.; and a controller for controlling the operations of the LEDs based on an input signal from the input unit, which is installed in the outer mask unit 2 or the like.

In addition, although not shown, the LED mask for personal combination stimulation according to an embodiment of the disclosure may include a charging unit (not shown) having a power supply terminal electrically connected to the charging terminal of the power supply unit, in a mounting frame to which the skin care mask is mounted.

Below, the effects of the LED mask for personal combination stimulation according to an embodiment of the disclosure will be described briefly.

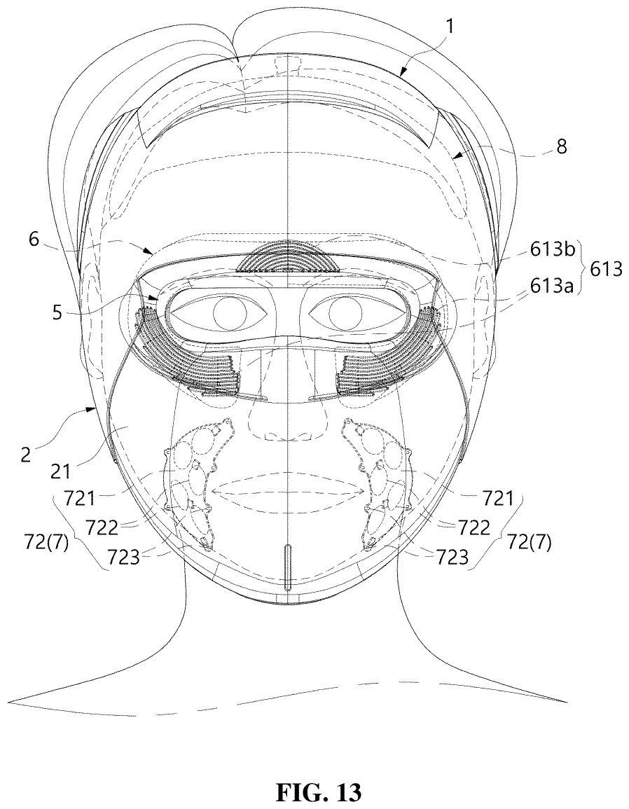

FIG. 13 is a view for illustrating the use state of an LED mask for personal combination stimulation according to an embodiment of the disclosure, in which the wearing state of the LED mask for personal combination stimulation is schematically illustrated.

Referring to FIGS. 2 to 13, when the secondary battery is charged with power received from a commercial power source while the skin care mask is being placed on the mounting frame (not shown), a user separates the skin care mask from the mounting frame and wears the skin care mask by fixing the wearing fixing means 3 to his/her head in the state that the light transfer supply means 6 is placed around the eyes and the inner mask unit 1 is in position to cover and seal the face.

Then, the combination LED modules 42 of the light irradiation unit 4 are turned on by controlling the input unit (not shown), so that the facial skin s to be cared can be irradiated with a light irradiance output of 10 mW/cm²±20% based on the light having a wavelength of 630 nm from the first LED chip and light having a wavelength of 850 nm from the second LED chip, thereby performing an improvement effect.

In this case, the light-blocking member 5, installed around the inner eye-exposure hole 12, and the outer eye-exposure hole 22 is formed of opaque silicone rubber, thereby blocking light emitted from the LEDs from entering the eyes when worn.

In addition, the light transfer supply means 6 installed in the inner mask unit 1 while being in contact with the light-blocking member 5 is formed of a light-transmitting material, and thus has the wrinkle improvement effect by irradiating the skin around the eyes with the light even though it is in close contact with the eye area, thereby solving the conventional problems. In more detail, it is possible to improve wrinkles around the eyes, which could not be performed in the conventional LED mask where light is not allowed to enter the skin around the eye area due to the opaque light-blocking member being in close contact with the eye area.

To describe more specifically the effects of improving wrinkles around the eyes by the foregoing light transfer supply means 6, light emitted from the combination LED modules 42 arranged on the substrate 41 of the light irradiation unit 4 enters the second side, i.e., the light guide tube connecting portion 612 through the first side of the opposite light guide tube 621, and the light passing through the light guide tube connecting portion 612 is transmitted to the flexible light-transmitting pad 611 formed of the light-transmitting material.

The light transmitted in this way is guided and scattered by the light supply line portions 613 of the flexible light-transmitting pad 611 and evenly diffused to the area between the eyebrows and the corners of the eyes on both sides, thereby effectively performing the wrinkle improvement.

In addition, the LED mask for personal combination stimulation according to an embodiment of the disclosure includes the nasolabial wrinkle improvement portion 7 provided at the nasolabial wrinkle formation area of the inner mask unit 1, thereby having the improvement effect on reducing the nasolabial wrinkles. In particular, the nasolabial wrinkle improvement portion 7 allows the LED light to be diffused while passing through the diffusion lens 722 of the wrinkle improvement plate 722 and evenly irradiated to the skin, thereby effectively improving the nasolabial wrinkles.

Meanwhile, the results of conducting clinical trials to verify the skin improvement effect of the LED mask for personal combination stimulation according to an embodiment of the disclosure will be briefly described (The following clinical trial was conducted at the request of the Department of Plastic Surgery, Hanyang University Guri Hospital, Protocol NO. Y&J_EC_P01).

Among subjects selected based on the selection/exclusion criteria, an experimental group was provided with the LED mask for personal combination stimulation according to the disclosure, and a control group was provided with an LED mask for personal combination stimulation (having the same appearance but using only one-tenth of the first LED chips (RED LED (630 nm)) without the second LED chips (IR LED (850 nm))).

During a clinical trial period, the subjects visited up to 6 times at the 4th, 8th, and 12th weeks of the application of a medical device for the clinical trial and at the 16th week for a tracking observation visit. After completing the 12-week application of the medical device for the clinical trial, the efficacy and safety were evaluated for 4 weeks.

The efficacy evaluation (specific details omitted) includes a crow's feet grading scale (CFGS) evaluation, a global aesthetic improvement scale (GAIS) evaluation for subjects' eye areas, etc. to be conducted by an independent evaluator, and the safety evaluation includes adverse effect checks, vital signs, and physical examination. In addition, areas to which the medical device for the clinical trial was applied were photographed at each visit, and the independent evaluator conducted the CFGS evaluation based on the photographs.

Among the planned number of subjects (26 subjects) selected through the given subject selection criteria (omitted), 25 subjects participated in the clinical trial, and the experimental group and the control group used the designated medical device for the clinical trial for 9 minutes per day, 5 times a week, and for 16 weeks.

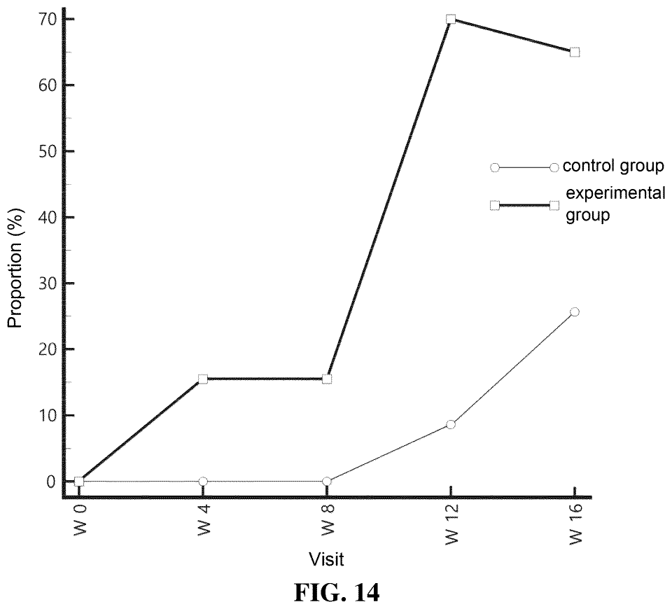

As above, when the results of the clinical trial were compared with respect to the proportion of the subjects improved in wrinkles around the eyes to compare the improvements in the CFGS, the same results were obtained as in Table 1 and their graphs were shown in FIG. 14.

TABLE 1

| | | Experimental group (N = 13) | | Control group (N = 12) | | |
|---|---|---|---|---|---|---|
| FAS | | N | % | N | % | p-value |
| 4 weeks | No change, D [symbol] | 11 | 84.6 | 12 | 100.0 | 0.480* |
| | Improvement | 2 | 15.4 | 0 | 0.0 | |
| 8 weeks | No change, D [symbol] | 11 | 84.6 | 12 | 100.0 | 0.480* |
| | Improvement | 2 | 15.4 | 0 | 0.0 | |
| 12 weeks | No change, D [symbol] | 4 | 30.8 | 11 | 91.7 | 0.004* |
| | Improvement | 9 | 69.2 | 1 | 8.3 | |
| 16 weeks | No change, D [symbol] | 5 | 35.8 | 9 | 75.0 | 0.111* |
| | Improvement | 8 | 61.5 | 3 | 25.0 | |
| p-value | | 0.031** | 1.000*** | 0.250** | 0.500*** | |
| | | Experimental group (N = 12) | | Control group (N = 12) | | |
| PPS | | N | % | N | % | p-value |
| 4 weeks | No change, D [symbol] | 10 | 83.3 | 12 | 100.0 | 0.478* |
| | Improvement | 2 | 16.7 | 0 | 0.0 | |
| 8 weeks | No change, D [symbol] | 10 | 83.3 | 12 | 100.0 | 0.478* |
| | Improvement | 2 | 16.7 | 0 | 0.0 | |
| 12 weeks | No change, D [symbol] | 3 | 25.0 | 11 | 91.7 | 0.003* |
| | Improvement | 9 | 75.0 | 1 | 8.3 | |
| 16 weeks | No change, D [symbol] | 4 | 33.3 | 9 | 75.0 | 0.100* |
| | Improvement | 8 | 66.7 | 3 | 25.0 | |

| | | | | |
|---|---|---|---|---|
| p-value | 0.031** | 1.000*** | 0.250** | 0.500*** |

*Fisher’s exact test,
**baseline vs. 16 weeks McNemar test,
**12 weeks vs. 16 weeks McNemar test [symbol]
indicates data missing or illegible when filed

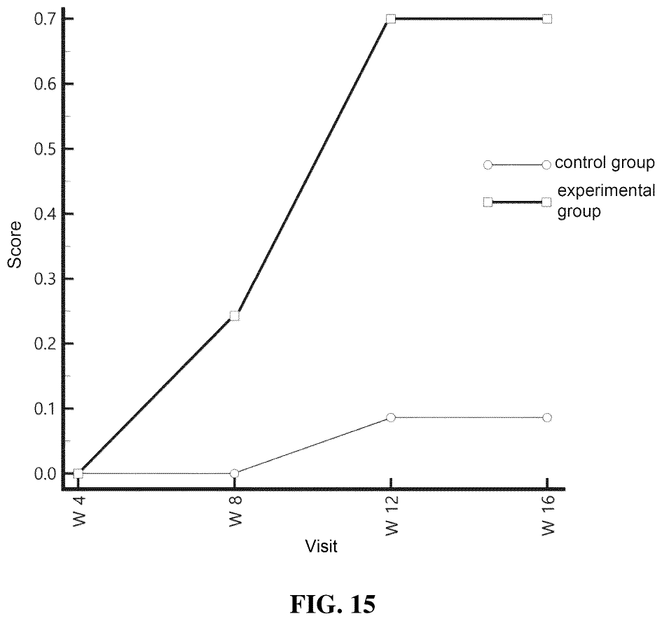

Referring to the comparison in the proportion between the subjects with improved wrinkles around the eyes in Table 1 (by an independent evaluator) and the graph of the comparison in the proportion between the subjects with improved wrinkles around the eyes in FIG. 14, a significant difference in score change between the experimental group and the control group was at 12 weeks after the initial application of the medical device for the clinical trial. Based on these results, the LED mask for personal combination stimulation according to the disclosure, was effective in improving the CFGS when used for 12 weeks. In Tables 1 and 2, Full Analysis Set (FAS) refers to a clinical trial subject group, Per Protocol Set (PPS) refers to clinical trial subjects who completed the clinical trial by complying well with the protocol among the subjects who belong to the FAS, and in FIGS. 14 and 15, W (WEEK) refers to the week.

Meanwhile, when the GAIS improvements in the eye area were compared by the comparison in the proportion between the subjects with improved wrinkles around the eyes based on the foregoing results of the clinical trial, the results were obtained as in Table 2 and their graphs were shown in FIG. 15.

TABLE 2

| Experimental group | | | | | Control group | | | | | |
|---|---|---|---|---|---|---|---|---|---|---|
| N | Mean | SD | Median | IQR | N | Mean | SD | Median | IQR | p-value |

FAS

| | | | | | | | | | | | |
|---|---|---|---|---|---|---|---|---|---|---|---|
| 4 weeks | 13 | 0.00 | 0.00 | 0.00 | 0 | 12 | 0.00 | 0.00 | 0.00 | 0 | 1.000* |
| 8 weeks | 13 | 0.23 | 0.44 | 0.00 | 1 | 12 | 0.00 | 0.00 | 0.00 | 0 | 0.220* |
| 12 weeks | 13 | 0.69 | 0.63 | 1.00 | 2 | 12 | 0.08 | 0.29 | 0.00 | 1 | 0.010* |
| 16 weeks | 13 | 0.69 | 0.63 | 1.00 | 2 | 12 | 0.08 | 0.29 | 0.00 | 1 | 0.010* |

| | | | | | | |
|---|---|---|---|---|---|---|
| p-value | 0.007** | 0.007** | 1.000*** | 0.317** | 0.317** | 1.000**** |

PPS

| | | | | | | | | | | | |
|---|---|---|---|---|---|---|---|---|---|---|---|
| 4 weeks | 12 | 0.00 | 0.00 | 0.00 | 0 | 12 | 0.00 | 0.00 | 0.00 | 0 | 1.000* |
| 8 weeks | 12 | 0.25 | 0.45 | 0.00 | 1 | 12 | 0.00 | 0.00 | 0.00 | 0 | 0.070* |
| 12 weeks | 12 | 0.75 | 0.62 | 1.00 | 2 | 12 | 0.08 | 0.29 | 0.00 | 1 | 0.004* |
| 16 weeks | 12 | 0.75 | 0.62 | 1.00 | 2 | 12 | 0.08 | 0.29 | 0.00 | 1 | 0.004* |

| | | | | | | |
|---|---|---|---|---|---|---|
| p-value | 0.007** | 0.007** | 1.000*** | 0.317** | 0.317** | 1.000**** |

*Mann-Whitney U test,
**baseline vs. 12 weeks Wilcoxon's signed-rank test,
***baseline vs. 16 weeks Wilcoxon's signed-rank test,
****12 weeks vs. 16 weeks Wilcoxon's signed rank test

Table 2 and FIG. 15 show the evaluation by a tester. The GAIS of the eye area was evaluated by the observations of the tester in person with the naked eyes without using the photographs based on 5 steps (−1 to 3 points) from −1 point (worse) to 3 points (very improved). The significant difference in the GAIS of the eye area between the experimental group and the control group was at 12 and 16 weeks. As a result, a degree of improvement in wrinkles around the eyes was confirmed with the naked eye when the LED mask for personal combination stimulation according to the disclosure is used for more than 12 weeks.

The terms “comprise,” “configure” and/or “have” specify the presence of stated components, unless there is a clearly different meaning in the disclosure, but do not preclude the presence thereof and should be construed to further include other components. Unless otherwise defined, all terms including technical or scientific terms used herein have the same meaning as commonly understood by those skilled in the art to which the disclosure pertains. General terms that are defined in a dictionary shall be construed to have meanings that are consistent in the context of the relevant art, and will not be interpreted as having an idealistic or excessively formalistic meaning unless clearly defined in the disclosure.

Although the configurations and operations of an LED mask for personal combination stimulation according to an embodiment of the disclosure have been described above, it will be understood by a person having ordinary knowledge in the art that the embodiments are merely illustrative and may be partially substituted and modified without departing from the scope of the disclosure.

Therefore, it should be understood that the scope of the disclosure falls within the disclosure defined in the appended claims and their equivalents.

### INDUSTRIAL APPLICABILITY

According to the disclosure, an LED mask for personal combination stimulation to be worn on a face is used for therapeutic purposes to treat damaged skin through selective regeneration or destruction of skin tissue, or for cosmetic purposes such as whitening on the principle that biochemical reactions are promoted within the skin as a combination stimulation LED where LED chips of various wavelength bands are combined is used to irradiate light to the skin of a human body.

1. A light emitting diode (LED) mask for personal combination stimulation, comprising:
a mask unit to be put on a face of a user; and
a light irradiation unit installed in the mask unit and comprising a plurality of LEDs arranged on a substrate, wherein
the LEDs comprise multiple types of LED chips to emit light of different wavelengths, and the multiple types of LED chips are configured as a combination LED module in a single support structure and arranged on the substrate.

2. The LED mask of claim 1, wherein the combination LED module comprises a first LED chip for a wavelength of 625 to 635 nm, and a second LED chip for a wavelength of 845 to 855 nm, which are configured in the single support structure.

3. The LED mask of claim 2, wherein
the first LED chip has a light irradiance output of 10 mW/cm²±20% for a wavelength of 630 nm, and the second LED chip has a light irradiance output of 10 mW/cm²±20% for a wavelength of 850 nm, and
the number of combination LED modules to be arranged ensures that the internal temperature of the mask unit rising due to heat generation caused by light emission does not exceed 41 to 43° C., and a distance between the combination LED module to be installed in the mask unit and the surface of the skin to be cared ensures that 50% of the light irradiation output is irradiated to the skin surface.

4. The LED mask of claim 3, wherein
the combination LED module has a light irradiation angle of 120°, and
the distance between the combination LED module 42 and the surface of the skin to be cared is 10 mm±10%.

5. The LED mask of claim 4, wherein the combination LED modules are installed on the substrate at intervals of 14 to 20 mm from each other taking a curvature of a facial area into account while ensuring that the internal temperature of the mask unit m does not exceed 41 to 43° C.

6. The LED mask of claim 1, wherein
the mask unit comprises an inner mask unit put on a user's face, formed with an inner eye-exposure hole, and comprising the light irradiation unit disposed on an outer surface, and an outer mask unit coupled to the inner mask unit and formed with an outer eye-exposure hole communicating with the inner eye-exposure hole, and
the LED mask further comprises:
a light-blocking member installed around the inner eye-exposure hole and the outer eye-exposure hole to prevent light from entering the eyes; and
a light transfer supply means installed in the inner mask unit to irradiate skin around the eyes with light, wherein the light transfer supply means comprises a light supply unit installed around the inner eye-exposure hole and formed of a light-transmitting material to make light enter and exit, and a light transfer unit arranged between the light supply unit and the LED to transfer light to the light supply unit.

7. The LED mask of claim 6, wherein
the light supply unit comprises a flexible light-transmitting pad seated on the inner side of the light-blocking member and having a communication hole to communicate with the inner eye-exposure hole, a light guide tube connecting portion formed on the flexible light-transmitting pad and having a fitting hole, and a plurality of light supply line portions formed on the flexible light-transmitting pad to move and diffuse light introduced through the light transfer unit,
the light transfer unit comprises a light guide tube having a first end disposed to face the LED of the light irradiation unit, and a second end connected to the fitting hole, and
the light-blocking member comprises an inner light-blocking unit formed in a substantially oval closed curve shape to be seated inside the circumference of the inner eye-exposure hole and having a lower center portion from which a nose-mounting portion having a nose-mounting groove is extended, and an outer light-blocking unit extended from the circumference of the inner light-blocking member at an acute angle to the inner light-blocking member and formed with a fitting protrusion at an edge thereof to be fitted to a gap between the inner eye-exposure hole and the outer eye-exposure hole

8. The LED mask of claim 7, wherein
the flexible light-transmitting pad comprises a seating pad portion arranged to surround an outer portion of the inner light-blocking unit, formed as a roughly oval body provided with a nose insertion cut portion to be seated around an eye area upon being worn, and comprising a seating protrusion formed along an inner edge thereof to allow the edge of the inner light blocking unit to be seated thereon; a front support portion extending downward on the seating pad portion being in contact with the nose insertion cut portion; a light guide tube installation portion bent at a bottom of the front support portion and formed with a light guide tube insertion port; and a cushion connection portion formed extending along an outer periphery of the seating pad portion, connected to the front support portion and fastened to the inner mask unit, and
the light supply line portion comprises an eyelid light supply line portion formed on the seating pad portion corresponding to both eyelids and end corners of the eyes to scatter and diffuse light, and an inter-eyebrow light supply line portion formed on the seating pad portion corresponding to an area between eyebrows.

9. The LED mask of claim 8, wherein
the eyelid light supply line portion is formed as multiple light-transmitting protrusion lines arranged in parallel from the surface of the seating pad portion being in contact with the nose insertion cut portion toward a second side, and
the inter-eyebrow light supply line portion is formed as multiple light-transmitting protruding lines repeatedly protruding in arc shapes from the seating pad portion corresponding to the inter-brow area and different in radius of curvature from each other.
